(19) United States
(12) Patent Application Publication
(10) Pub. No.: US 2026/0069223 A1
(43) Pub. Date: Mar. 12, 2026

(54) Magnetic Resonance Apparatus with a Monitoring User Interface and a Patient Interface

(71) Applicant: Siemens Healthineers AG, Forchheim (DE)

(72) Inventors: Leon Wildt, Erlangen (DE); Daniel Rinck, Forchheim (DE); Sebastian Schroth, Erlangen (DE)

(73) Assignee: Siemens Healthineers AG, Forchheim (DE)

(21) Appl. No.: 19/324,470

(22) Filed: Sep. 10, 2025

(30) Foreign Application Priority Data
Sep. 10, 2024 (DE) 10 2024 208 577.4

(51) Int. Cl. A61B 5/00; A61B 5/055; G01R 33/28; G01R 33/56

(52) U.S. Cl. CPC A61B 5/7465; A61B 5/0077; A61B 5/055; G01R 33/283; G01R 33/5608

(57) ABSTRACT

The disclosure relates to a magnetic resonance apparatus with a scanner, a patient receiving area surrounded at least in part by the scanner, a monitoring user interface in which monitoring and/or control of a magnetic resonance examination may be performed by a user, a patient interface, which is arranged within the scanner, and a data transceiver configured to transmit data between the patient interface and the monitoring user interface. The data transceiver may include at least one cable unit. Within the scanner, the at least one cable unit may include optical fibers.

### CROSS REFERENCE TO RELATED APPLICATIONS

This patent application claims priority to German Patent Application No. 102024208577.4, filed Sep. 10, 2024, which is incorporated herein by reference in its entirety.

### BACKGROUND

The present disclosure relates to a magnetic resonance apparatus with a scanner, a patient receiving area surrounded at least in part by the scanner, a monitoring user interface, by means of which the monitoring and/or control of a magnetic resonance examination is undertaken by a user, a patient interface, which is arranged within the scanner, and a data transmission unit, which may be configured for transmission of data between the patient interface and the monitoring user interface.

During a magnetic resonance examination, the patient is located in a patient receiving area of a scanner of a magnetic resonance apparatus. The scanner is arranged within an examination space, wherein the examination space may be screened off from the outside in respect of RF radiation. Medical operating personnel, for example a doctor in charge of the magnetic resonance examination, on the other hand are located during the magnetic resonance examination in a control room, which may be separated from the examination space. An exchange of information between the patient and the medical operating personnel during a magnetic resonance examination is therefore only possible to a restricted extent. Since moreover, magnetic resonance examinations can take a longer time, for example from 20 minutes up to an hour, an exchange of information between the patient and the medical operating personnel and/or between an environment of the patient and the medical operating personnel is sensible and desirable.

In previous magnetic resonance apparatuses, an exchange of information between the patient and the medical operating personnel during a magnetic resonance examination was conducted by means of a patient call ball. A patient call ball may comprise an area that the patient can press in an emergency situation or if they feel unwell. A signal triggered by pressing the area is conducted outwards via a compressed air hose with which the patient call ball is connected and transmitted to a user interface that is arranged within the control room, for output to the medical operating personnel. However, the disadvantage of such an air hose is that, during movements of the patient table, for example when the patient table is moved into the patient receiving area or when the patient table is moved out of the patient receiving area, damage, for example squashing and/or pinching of said air hose, can occur.

A further option for an exchange of information between the patient and the medical operating personnel during a magnetic resonance examination is known for example from US 2017 0 127 053 A1. Here information for the patient is shown on a display that is arranged on a wall of the examination space and by means of a mirror the information is made visible for the patient by being projected within the patient receiving area. Here however, a size of the information shown varies depending on the distance between the mirror and the display. Moreover, such an arrangement restricts an area available to the patient within the patient receiving area, so that in addition this can give rise to patient anxiety during the magnetic resonance examination.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 shows a magnetic resonance apparatus, according to an exemplary embodiment, including a monitoring user interface and a patient interface.

FIG. 2 shows a patient receiving area of the magnetic resonance apparatus with a first interface unit of the patient interface, according to an exemplary embodiment.

FIG. 3 shows the patient receiving area with the first interface unit in a cross-sectional diagram, according to an exemplary embodiment.

FIG. 4 shows the patient receiving area of the magnetic resonance apparatus with a second interface unit of the patient interface, according to an exemplary embodiment.

The exemplary embodiments of the present disclosure will be described with reference to the accompanying drawings. Elements, features and components that are identical, functionally identical and have the same effect are - insofar as is not stated otherwise - respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An object of the present disclosure is to provide a space-saving and robust technology for an exchange of information between the patient or an environment of the patient and a member of the medical operating personnel.

The disclosure is based on a magnetic resonance apparatus with a scanner, a patient receiving area at least partly surrounded by the scanner, a monitoring user interface, by means of which a monitoring and/or control of a magnetic resonance examination is undertaken by a user, a patient interface, which is arranged within the scanner, and a data transmission unit, which may be configured for transmission of data between the patient interface and the monitoring user interface. It is proposed that the data transmission unit may comprise at least one cable unit wherein, within the scanner, the at least one cable unit may comprise optical fibers.

The magnetic resonance apparatus may comprise a medical and/or diagnostic magnetic resonance apparatus, which may be designed and/or embodied to acquire medical and/or diagnostic image data, such as medical and/or diagnostic magnetic resonance image data of a patient. To this end, the magnetic resonance apparatus may comprise the scanner. The scanner may comprise a magnet unit for acquiring the medical and/or diagnostic image data. Advantageously, the scanner may comprise the magnet unit, a basic magnet, a gradient coil unit and a radio-frequency antenna unit. The radio-frequency antenna unit may be arranged permanently within the scanner and may be designed and/or arranged for emitting an excitation pulse.

The basic magnet may be configured for creating a homogeneous basic magnetic field with a defined magnetic field strength, such as with a magnetic field strength of 3 T or 1.5 T etc. The basic magnet may be configured to create a strong and constant basic magnetic field. The homogeneous basic magnetic field may be arranged or to be found within the patient receiving area of the magnetic resonance apparatus. The gradient coil unit may be configured to create magnetic field gradients, which are used for a spatial encoding during an imaging process.

The patient receiving area may be designed and/or embodied for receiving the patient, in particular the region of the patient to be examined, for a medical magnetic resonance examination. The patient receiving area may comprise that area that is available to the patient during a magnetic resonance examination. To this end, for example the patient receiving area may be configured in a cylindrical shape and/or is surrounded in a cylindrical shape by the scanner, in particular the magnet unit, of the magnetic resonance apparatus.

A Field of View (FoV) and an isocenter of the magnetic resonance apparatus is arranged within the patient receiving area. The FoV may comprise an acquisition region of the magnetic resonance apparatus, within which the conditions for an acquisition of medical image data, especially of magnetic resonance image data, are present within the patient receiving area, such as a homogeneous basic magnetic field. The isocenter of the magnetic resonance apparatus may comprise the region and/or point within the magnetic resonance apparatus that has the optimal and/or ideal conditions for the acquisition of medical image data. The isocenter may comprise the most homogeneous magnetic field area within the magnetic resonance apparatus.

During the magnetic resonance examination on the patient an exchange of information between the medical operating personnel and the patient takes place between the monitoring user interface and the patient interface. In an exemplary embodiment, the patient may be located during the magnetic resonance examination within the patient receiving area and thus within the examination space in which the scanner of the magnetic resonance apparatus is arranged. The medical operating personnel on the other hand are located during the magnetic resonance examination for control and monitoring of the magnetic resonance examination within the control room, which may be screened off from the examination space in respect of radio-frequency radiation.

Visual information can be transmitted by means of the data transmission unit from the monitoring user interface to the patient interface and output visually there for a patient. To this end, the patient interface can also have an output unit, in particular a visual output unit. Moreover, patient information can be acquired by means of the patient interface and transmitted to the monitoring user interface and output there for a user. To this end the patient interface can also have an acquisition unit, for example a sensor unit and/or a camera unit, for acquisition of patient information.

The monitoring user interface can also have an input unit, by means of which a user can enter information for the patient. Such an input unit may comprise a microphone and/or a keyboard and/or a computer mouse. The monitoring user interface can also comprise a processor, which automatically, for example, on the basis of an examination context, transfers information to the patient interface for output to the patient. Moreover, the monitoring user interface may comprise an output unit, which may be configured to output patient information acquired by means of the patient interface. For example, the output unit can comprise a visual output means, such as, in particular, a display. The monitoring user interface, for controlling a data transmission between the monitoring user interface and the patient interface, can also have a controller.

The data transmission unit (data transceiver) may be configured to transmit data between the monitoring user interface and the patient interface. In an exemplary embodiment, the data transmission may be embodied for a bidirectional data transmission between the monitoring user interface and the patient interface. To this end the data transmission unit has at least one cable unit, which within the scanner, may comprise optical fibers. In an exemplary embodiment, the data transmission unit has a number of cable units, which within the scanner, comprise optical fibers. In this way it can be ensured that an exchange of information between the patient interface and the monitoring user interface may be only undertaken within the scanner by means of optical fibers.

Outside the scanner and also outside the examination space the cable unit can comprise cables other than optical fibers. Moreover, the data transmission unit, outside the scanner and/or outside the examination space, can also have further data transmission elements appearing sensible to the person skilled in the art.

Advantageously the disclosure enables there to be a trouble-free exchange of information between a patient and the medical operating personnel during a magnetic resonance examination. In particular in this way both any adverse effect on the data transmission by the magnet unit and also any adverse effect on the magnetic resonance examination by the data transmission unit and/or the patient interface can be prevented. Moreover, there can be a low-cost data exchange between the monitoring user interface and the patient interface within the scanner by means of the optical fibers.

In an advantageous development of the inventive magnetic resonance apparatus there can be provision for the patient interface to comprise at least two different interface units and for the data transmission unit to comprise at least two cable units with optical fibers, wherein each of the at least two interface units may be connected by one of the at least two cable units in each case for a transmission of data to the monitoring user interface. In an exemplary embodiment, the at least two different interface units of the patient interface can comprise units and/or means for providing and/or outputting the information for the patient, such as an output unit. An output unit can for example comprise a display or a projection unit or a unit for output and/or emission of light etc. Moreover, the at least two different interface units can also comprise units and/or means for acquiring the information from the patient and/or from an environment of the patient, such as an input unit and/or an acquisition unit. An input unit may be configured for input of information by the patient, such as a microphone or a key and/or a pushbutton etc. An acquisition unit may be configured for an automatic acquisition of information during the magnetic resonance examination, such as a camera system and/or movement sensors and/or temperature sensors etc.

This embodiment of the disclosure makes possible a simple type of data transmission between an area within the scanner, in particular the patient receiving area, and an area outside the scanner, in particular the control room, for different applications and/or purposes. Moreover, in this way a simple separation of different data to be transmitted, such as sensor data and/or camera data and/or information to the patient, can be achieved. Moreover, in such a way, a simple separation between incoming and outgoing data can be achieved by means of the at least two cable units.

In an advantageous development of the inventive magnetic resonance apparatus there can be provision for the data transmission unit to have at least one signal converter, which may be embodied for conversion of optical signals into electronic signals and/or from electronic signals into optical signals. The signal converter may comprise optical elements, such as optical lenses and/or an optical lens system and/or prisms and/or further elements appearing sensible to the person skilled in the art. The at least one signal converter can be embodied for coupling data and/or signals into the optical fibers of the cable units and/or for coupling data and/or signals out from the optical fibers of the cable unit. In an exemplary embodiment, a signal converter can be available for different data and/or cable units in each case. In this way there can advantageously be an exchange of data between units with different data inputs.

In an advantageous development of the inventive magnetic resonance apparatus there can be provision for the data transmission unit to have a common signal converter for a conversion of optical signals into electronic signals and from electronic signals into optical signals for at least two cable units of the data transmission unit. In this way an especially compact and component-saving construction of the data transmission unit can be provided. In particular in this way the same infrastructure for data transmission of different data between the patient interface and the monitoring user interface can be used and thus components and costs advantageously saved.

In an advantageous development of the inventive magnetic resonance apparatus there can be provision for the patient interface to have an interface unit, which may comprise a camera system for patient monitoring with two or more camera units, wherein one cable unit of the data transmission unit may be assigned to the camera system. Each of the two or more camera units may comprise at least one camera. The individual camera units, especially the individual cameras, are arranged within the patient receiving area of the scanner. In an exemplary embodiment, the individual camera units, especially the individual cameras, are arranged on a housing surrounding the patient receiving area. In an exemplary embodiment, the individual camera units, especially the individual cameras, are also integrated into the housing surrounding the patient receiving area, so that a surface planar with the patient receiving area is present. The cable unit of the data transmission unit assigned to the camera system may be connected to the camera system and may be (e.g., exclusively) configured for a transmission of camera data of the camera system.

This embodiment of the disclosure makes it possible to capture the patient during a magnetic resonance examination from different positions. In an exemplary embodiment, a larger overall image, which can be assembled for camera data of the two or more camera units, may be obtained. This makes possible advantageous monitoring of the patient during a magnetic resonance examination by the medical operating personnel, so that with this the patient's safety can be enhanced. In an exemplary embodiment, a critical situation of the patient and/or the patient being unwell can be recognized during the magnetic resonance examination by the medical operating personnel and remedial measures can be initiated. A critical situation of the patient during a magnetic resonance examination can for example be an undesired contact between the patient and a housing surrounding the patient receiving area or also contact between the arms and/or hands and other parts of the body. A further advantage of this embodiment is that there can be a simple data transmission within the scanner by means of the cable unit, and moreover any adverse effect on the magnetic resonance examination by data transmission can advantageously be prevented.

In an advantageous development of the inventive magnetic resonance apparatus there can be provision for the two or more camera units to be arranged spaced apart from one another in the longitudinal direction of the patient receiving area. The longitudinal direction of the patient receiving area may be parallel to a direction in which the patient table is moved into the patient receiving area and/or aligned in parallel to a magnetic flux density of the basic magnetic field. This embodiment likewise makes it possible to capture the patient during a magnetic resonance examination from different positions, especially from different positions in the longitudinal direction of the patient receiving area, within the patient receiving area. In an exemplary embodiment, a larger overall image of the patient, which can be assembled from camera data of the two or more camera units, can be determined and an advantageous patient monitoring thereby be provided.

In an advantageous development of the inventive magnetic resonance apparatus there can be provision for the camera system to comprise at least three camera units, wherein two neighboring camera units are arranged in each case essentially at the same distance from one another in the longitudinal direction of the patient receiving area. Essentially two neighboring camera units being at the same distance from one another in the longitudinal direction is especially to be understood, as a distance between two respective neighboring camera units having a maximum deviation of ±10 cm in relation to a distance between two further neighboring camera units. In an exemplary embodiment, the distance between two neighboring camera units has a maximum deviation of ±5 cm and especially advantageously a maximum deviation of ±3 cm in relation to a distance between two further neighboring camera units. In an exemplary embodiment, the camera system has four camera units, wherein two neighboring camera units are arranged in each case at essentially the same distance from one another in the longitudinal direction. In this way the individual camera units are distributed in the longitudinal direction within the patient receiving area, so that, with the aid of image data of the individual camera units, advantageously an overall image of the patient located within the receiving area can be created. This enables a monitoring of the patient during a magnetic resonance examination to be undertaken by the medical operating personnel and thus the safety of the patient to be enhanced. In an exemplary embodiment, a critical situation of the patient and/or the patient being unwell can be recognized during the magnetic resonance examination by the medical operating personnel and remedial measures can be initiated.

In an advantageous development of the inventive magnetic resonance apparatus there can be provision for each of the two or more camera units to have at least one 3D camera in each case or at least two 2D cameras in each case. The 3D camera can for example comprise a depth camera or a TOF camera etc. The at least two 2D cameras may comprise an RGB camera and/or an infrared camera and/or a further 2D camera appearing sensible to the person skilled in the art. This advantageously enables 3D image data for monitoring the patient to be provided, which makes it possible for a member of the medical operating personnel looking after the magnetic resonance examination to recognize a facial expression of the patient during a magnetic resonance examination and thus also an emotional state of the patient. The 3D image data can be acquired, directly by means of the 3D camera, or established by the 2D image data.

In an exemplary embodiment, the individual cameras, especially the 3D cameras or the 2D cameras, are arranged in a screened housing within the scanner, especially within the patient receiving area, so that any undesired interaction between the cameras and the scanner can advantageously be prevented.

In an advantageous development of the inventive magnetic resonance apparatus there can be provision for the at least two 2D cameras each to have a camera unit distributed in the circumferential direction around the patient receiving area on a housing surrounding the patient receiving area, wherein the at least two 2D cameras a camera unit in the longitudinal direction of the patient receiving area each have the same position. The circumferential direction runs and/or extends, in parallel to a circular base surface of the cylindrical patient receiving area on the housing. In an exemplary embodiment, each of the two or more camera units has a defined position in the longitudinal direction of the patient receiving area on the housing surrounding the patient, wherein the individual 2D cameras of the respective camera unit also have a defined position in the longitudinal direction of the patient receiving area. In this way a three-dimensional image of the patient can be created at each position of camera unit, wherein the three-dimensional image of the patient is formed from individual 2D images of the at least two 2D cameras of the respective camera unit. In an exemplary embodiment, three-dimensional images can be computed with aid of the beam set theory. In this way, an especially low-cost acquisition of monitoring data of the patient and/or of the patient receiving area can be provided by means of the 2D cameras.

In an advantageous development of the inventive magnetic resonance apparatus there can be provision for each of the two or more camera units each to have three 2D cameras, wherein the three 2D cameras each have a camera unit arranged in the circumferential direction around the patient receiving area distributed on a housing surrounding the patient receiving area and having the same position in the longitudinal direction of the patient receiving area. In this way a 2D image can be captured in each case from different directions and a 3D image can be established from the different 2D mages of a camera unit. This makes possible a complete capture of the patient within the patient receiving area, so that critical situations of the patient, such as any undesired contact between the patient and a housing surrounding the patient receiving area or also contact between the arms and/or hands and further parts of the body etc. can be detected.

In an advantageous development of the inventive magnetic resonance apparatus there can be provision for the first 2D camera to be arranged at the top in the middle of the housing surrounding the patient receiving area and for a second 2D camera and a third 2D camera each to be arranged on a side area of the housing surrounding the patient receiving area, wherein the second 2D camera and the third 2D camera are arranged on opposite side areas. The first 2D camera here may be essentially arranged at a highest point of the patient receiving area on the housing surrounding the patient receiving area. The two side areas on which the second 2D camera and the third 2D camera are arranged are located during a magnetic resonance examination to the side next to a patient located within the patient receiving area. The arrangement of the second 2D camera and the third 2D camera on opposite side areas on the housing surrounding the patient receiving area thus enables the patient to be captured from both sides by a 2D camera.

In particular this embodiment of the disclosure enables a region of the patient located within the patient receiving area to be captured completely. This for example makes possible live tracking of the patient for a member of the medical operating personnel at the monitoring user interface. Moreover, in this way, a constant check that SAR limit values are being adhered to can be performed. There can also be monitoring in respect of a movement of the patient during the magnetic resonance examination and/or a check on the position of the patient and/or an imaging of a local radio-frequency coil etc. through the camera data provided.

In an advantageous development of the inventive magnetic resonance apparatus there can be provision for the second 2D camera and the third 2D camera to have a position with a same height on the housing surrounding the patient receiving area and to be arranged with an essentially maximum distance from one another on the housing surrounding the patient receiving area. In particular here the second 2D camera and the third 2D camera are arranged at the same height in relation to a support surface for supporting and/or positioning the patient. In an exemplary embodiment, the second 2D camera and the third 2D camera are arranged in such a way on the housing surrounding the patient receiving area that the second 2D camera and the third 2D camera are each located to the side next to the patient at around half the height of the patient receiving area on the patient receiving area. Thus the second 2D camera and the third 2D camera are arranged in the horizontal direction, which may be aligned vertically to the longitudinal extent of the patient receiving area, in a widest area of the patient receiving area, so that the second 2D camera and the third 2D camera are arranged with a maximum distance from one another at the same position in the longitudinal direction of the patient receiving area on the housing surrounding the patient receiving area.

This likewise enables a region of the patient located within the patient receiving area to be completely captured. This makes possible live tracking of the patient at the monitoring user interface for a member of the medical operating personnel. Moreover, in this way a constant check that SAR limit values are being adhered to can be performed. There can also be monitoring in respect of a movement of the patient during the magnetic resonance examination and/or a check on the position of the patient and/or a capture of a local radio-frequency coil etc. through the camera data provided.

In an advantageous development of the inventive magnetic resonance apparatus there can be provision for the magnetic resonance apparatus to have a processor, wherein the processor may be embodied:
to establish a three-dimensional image with the aid of two-dimensional images of the 2D cameras of a camera unit, and/or
to establish a three-dimensional overall image and/or a patient model from three-dimensional images of at least two camera units.

The processor may be arranged outside the scanner. In an exemplary embodiment, the processor may be also arranged outside the examination space. The processor may be arranged within the control room. The processor may include the monitoring user interface, which prepares an evaluation of the captured camera data for an output to the monitoring user interface.

The processor may comprise at least one processing module and/or a processor. In this way the processor may be embodied in particular to execute computer-readable instructions. In an exemplary embodiment, the processor can also comprise a memory unit, wherein computer-readable information may be stored on the memory unit, wherein the processor may be embodied to load the computer-readable information from the memory unit and to execute the computer-readable information. The components of the processor can predominantly be embodied in the form of software components. These components may also be realized in part, in particular when it is a matter of especially fast calculations, in the form of software-supported hardware components, for example FPGAs or the like. Likewise, the interfaces needed, for example, when it is only a matter of accepting data from other software components, can be embodied as software interfaces. They can also be embodied as hardware interfaces however, which are activated by suitable software. Naturally, it is also conceivable for a number of the said components to be realized in the form of an individual software component or to be assembled from software-supported hardware components.

In an exemplary embodiment, the processor, for establishing three-dimensional images, has an algorithm, which for example may comprise a RANSAC (RANdom SAmple Consensus) algorithm and/or an ICP (Iterative Closest Point) algorithm.

This enables 3D image data of the patient to be provided during the magnetic resonance examination, which, for example, makes possible a live position tracking of the patient for a member of the medical operating personnel at the monitoring user interface. Moreover, it can also be monitored in this way whether the patient is correctly carrying out instructions received during the magnetic resonance examination.

In an advantageous development of the inventive magnetic resonance apparatus there can be provision for the individual 3D cameras or the 2D cameras of the respective camera unit to have a fixed field of view, wherein the fields of view of directly neighboring camera units at least partly overlap. In an exemplary embodiment, the fields of view of the individual cameras, especially of the individual 3D cameras or of the individual 2D cameras, are embodied as equal in size. In this way advantageously a complete area without interruptions within the patient receiving area can be captured by means of the camera system. A further advantage of this embodiment is that there can be a complete monitoring of the position of the patient within the patient receiving area.

In an advantageous development of the inventive magnetic resonance apparatus there can be provision for the patient interface to comprise an interface unit with at least one illumination element, wherein the at least one illumination element may be assigned to a cable unit of the data transmission unit. The at least one illumination element may be configured to illuminate the patient receiving area during a magnetic resonance examination. To this end the at least one illumination element may be embodied as a passive illumination element, which may be exclusively embodied to emit light, wherein a coupling in and/or transmission of light signals to the at least one passive illumination element may be undertaken by means of the cable unit with the optical fibers. The at least one illumination element, especially the at least one passive illumination element, may be arranged on the housing surrounding the patient receiving area.

The magnetic resonance apparatus, such as an illumination unit of the magnetic resonance apparatus, can moreover have at least one further illumination element. This at least one further illumination element may comprise an active illumination element, in particular a light-generating illumination element. Such light-generating illumination elements are arranged on the other hand outside the patient receiving area and/or outside the scanner. In an exemplary embodiment, light signals are transmitted by means of the cable unit, in particular the optical fibers, into the patient receiving area and this may be illuminated by means of the passive illumination elements. The cable unit of the data transmission unit assigned to the illumination unit can be embodied exclusively for transmission of light signals of the illumination unit.

This embodiment of the disclosure has the advantage that the patient receiving area can be illuminated especially easily. Moreover, monitoring of the patient, especially by means of the camera systems can be made easier by this, since contours of the patient and/or emotions of the patient and/or a facial expression of the patient can be recognized especially easily in the camera data by the illumination of the patient receiving area.

The illumination unit may be controlled by means of a controller of the monitoring user interface, so that the medical operating personnel can control an illumination and/or lighting up of the patient receiving area from the control room by means of the monitoring user interface. In an exemplary embodiment, a lighting up and/or illumination of the patient receiving area can be adapted to an emotional state of the patient and/or to individual situations in the examination workflow.

In an advantageous development of the inventive magnetic resonance apparatus there can be provision for the patient interface to have an interface unit with a visual output unit for output of information to the patient, wherein the visual output unit may be assigned a cable unit of the data transmission unit. The cable unit assigned to the visual output unit of the data transmission unit can be embodied exclusively for transmission of output data of the visual output unit. The visual output unit may comprise a visual output element, which may be arranged within the patient receiving area during a magnetic resonance examination. In an exemplary embodiment, the visual output element can be arranged on the housing surrounding the patient receiving area. As an alternative to this, the patient table can also comprise a holder in a head area of the patient table, wherein the visual output element can be arranged on this holder, so that the visual output element may be moved with the patient as well during a movement of the patient table and thus of the patient and thus is always arranged in a field of view of the patient. The visual output element can comprise a display. In an exemplary embodiment, the visual output element can also comprise a passive display, so that any interaction by the visual output element with the scanner is advantageously prevented during the magnetic resonance examination. In a further embodiment of the visual output element, said unit can comprise a projection surface. Information and/or instructions can be transferred from the medical operating personnel to the patient during the magnetic resonance examination by means of the visual output unit.

In an advantageous development of the inventive magnetic resonance apparatus there can be provision for the visual output unit to comprise a projection unit with a projection surface and an optical projection element. The projection surface can be or can comprise a mirror or a defined projection surface, such as the housing surrounding the patient receiving area. The optical projection elements can for example comprise a lens and/or a lens system and/or a prism etc. In this way an introduction of information, in particular a blending in of information, into the patient receiving area during the magnetic resonance examination can be realized by means of pure optical data transmission. As a consequence, any undesired interaction with the magnet unit can advantageously be prevented.

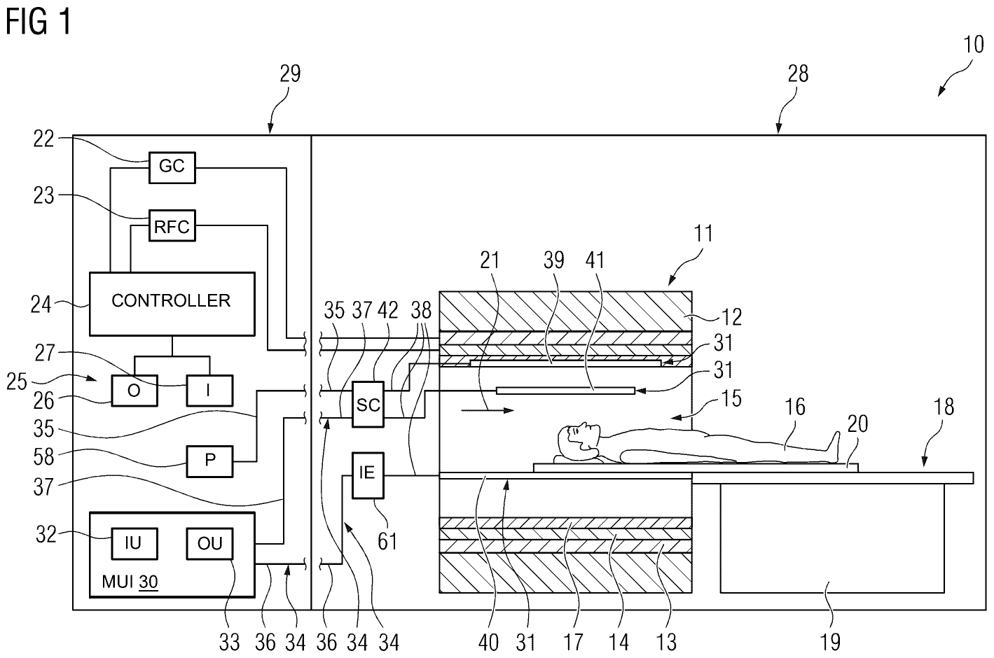

Shown schematically in FIG. 1 is a magnetic resonance apparatus 10. The magnetic resonance apparatus 10 may comprise a scanner/scanning unit (scanner) embodied as a magnet unit 11. The scanner, especially the magnet unit 11, may comprise a basic magnet 12, a gradient coil unit 13 and a radio-frequency antenna unit 14. Moreover, the magnetic resonance apparatus 10 has a patient receiving area 15 for receiving a patient 16 for a magnetic resonance examination. The patient receiving area 15 may be embodied in an exemplary embodiment in a cylindrical shape and may be surrounded in a circumferential direction by the magnet unit 11. However, an embodiment of the patient receiving area 15 that differs from this is always conceivable. The scanner, especially the magnet unit 11, moreover has a housing 17 surrounding the patient receiving area 15, especially a cylindrical housing 17.

For a positioning of the patient 16, especially of a region of the patient 16 to be examined, within the patient receiving area 15, the magnetic resonance apparatus 10 has a patient support apparatus 18. The patient support apparatus 18 has a base 19 and a patient table 20 able to be moved in relation to the base 19. The patient table 20 may be embodied, for a positioning of the patient 16, especially of the region of the patient 16 to be examined, movably within the patient receiving area 15. In particular here the patient table 20 may be supported for movement in the direction of a longitudinal extent of the patient receiving area 15 and/or in the z direction.

The basic magnet 12 of the magnet unit 11 may be embodied for creating a strong and especially constant basic magnetic field 21. In an exemplary embodiment, the basic magnet 12 can be embodied for example as a superconducting basic magnet 12 or also as a permanent magnet. The gradient coil unit 13 of the magnet unit 11 may be embodied for creation of magnetic field gradients, which are used for spatial encoding during an imaging. The gradient coil unit 13 may be controlled by means of a gradient controller 22 of the magnetic resonance apparatus 10. The radio-frequency antenna unit 14 of the magnet unit 11 may be embodied for excitation of a polarization, which occurs in the basic magnetic field 21 created by the basic magnet 12. The radio-frequency antenna unit 14 may be controlled by a radio-frequency antenna controller 23 of the magnetic resonance apparatus 10 and emits radio frequency magnetic resonance sequences into the patient receiving area 15 of the magnetic resonance apparatus 10.

For control of the basic magnet 12, the gradient controller 22 and for control of the radio-frequency antenna controller 23 the magnetic resonance apparatus 10 has a system controller 24. The system controller 24 centrally controls the magnetic resonance apparatus 10, such as the carrying out of a predetermined imaging gradient echo sequence. Moreover, the system controller 24 may comprise an evaluation unit (e.g., processor, processing circuitry), not shown in any greater detail, configured to evaluate data, such as medical image data, that may be acquired during the magnetic resonance examination.

Furthermore, the magnetic resonance apparatus 10 may comprise a user interface 25, which may be connected to the system controller 24. Control information such as imaging parameters, as well as reconstructed magnetic resonance images, can be shown on a display unit 26, for example on at least one monitor and/or a display of the user interface 25 for a member of the medical operating personnel. Furthermore, the user interface 25 has an input unit 27, by means of which information and/or parameters can be entered during a measuring process by one of the medical operating personnel.

The scanner of the magnetic resonance apparatus 10 may be arranged, together with the patient support apparatus 15, within an examination space 28. The system controller 24 on the other hand may be arranged, together with the user interface, within a control room 29. The control room 29 may be embodied separately from the examination space 28. In particular the examination space 28 may be screened off in respect of radio frequency radiation from the control room 29. During a magnetic resonance examination, the patient 16 may be located within the examination space 28, the medical operating personnel on the other hand are located within the control room 29 for monitoring and control of the magnetic resonance examination.

For an exchange of information between the medical operating personnel and the patient 16 during the magnetic resonance examination the magnetic resonance apparatus 10 has a monitoring user interface 30 and a patient interface 31. The monitoring user interface 30 may be arranged within the control room 29. The monitoring user interface 30 may be embodied for monitoring and/or control of the patient 16 during the magnetic resonance examination. To this end the monitoring user interface 30 has an input unit 32 and an output unit 33. The input unit 32 may have at least one input element (e.g., a number of input elements) such as a microphone and/or a keyboard and/or a computer mouse etc. The output unit 33 has at least one output element, such as a monitor.

The patient interface 31 on the other hand may be located within the examination space 28. In an exemplary embodiment, the patient interface 31 may be arranged within the patient receiving area 15.

For a data transmission between the monitoring user interface 30 and the patient interface 30 the magnetic resonance apparatus 10 has a data transmission unit 34. The data transmission unit 34 has at least one cable unit 35, 36, 37, wherein the at least one cable unit 35, 36, 37 within the scanner, especially the magnet unit 11, may comprise optical fibers 38 for data transmission between the monitoring user interface 30 and the patient interface 31. In an exemplary embodiment, the data transmission between the monitoring user interface 30 and the patient interface 31 within the magnet unit 11 takes place exclusively via optical fibers 38 in order to avoid any undesired interaction with the magnet unit 11.

The patient interface 31 has at least two interface units 39, 40, 41, wherein the data transmission unit 34 too may comprise at least two cable units 35, 36, 37 each with optical fibers 38. In an exemplary embodiment the patient interface 31 may comprise three different interface units 39, 40, 41 and the data transmission unit three cable units 35, 36, 37 each with optical fibers 38. In an exemplary embodiment, each of the three interface units 39, 40, 41 may be connected to one of the three cable units 35, 36, 37 for transmission of data to the monitoring user interface 30.

The data transmission unit (data transceiver) 34 may comprise a signal converter 42, which may be embodied for conversion of optical signals into electronic signals and/or of electronic signals into optical signals. In an exemplary embodiment, the data transmission unit 34 has a single signal converter 42 for all cable units 35, 37 of the data transmission unit 34, provided a signal conversion may be necessary for the data and/or signals to be transmitted. As an alternative to this, the data transmission unit can have a separate signal converter 42 for each of the cable units 35, 37.

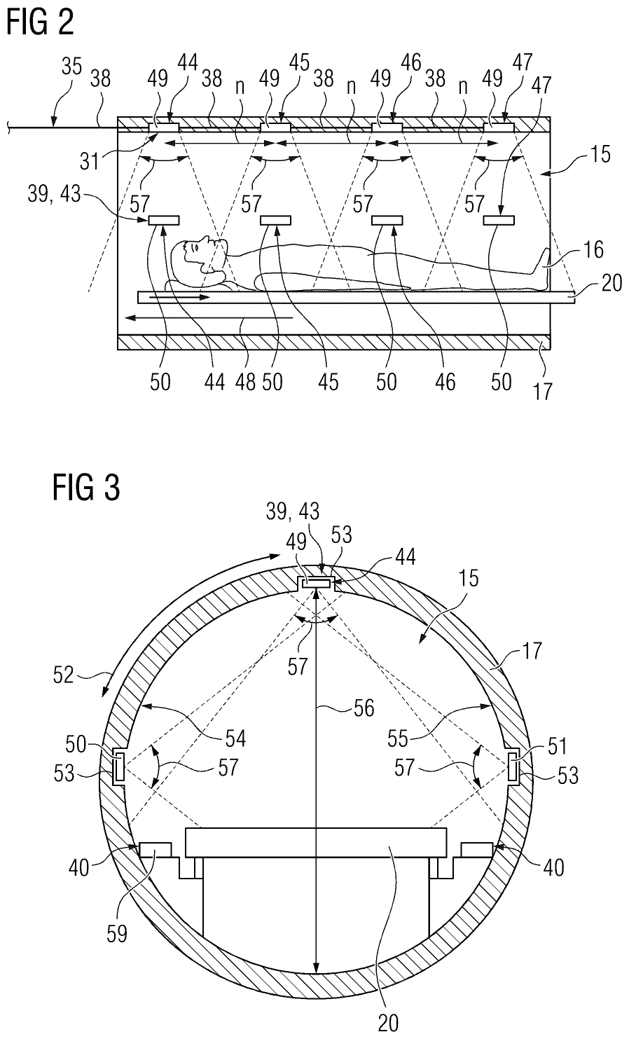

A first interface unit (interface, input/output interface) 39 of the patient interface 30 is shown in FIGS. 2 and 3 in greater detail. The first interface unit 39 may comprise a camera system 43 for patient monitoring, wherein the first interface unit 39 may be connected to the camera system 43 with a first cable unit 35 of the data transmission unit 34 for a data transmission from the patient interface 31, especially the camera system 43, to the monitoring user interface 30. The camera system 43 may comprise two or more camera units 44, 45, 46, 47. In an exemplary embodiment, the camera system 43 may comprise four camera units 44, 45, 46, 47. The four camera units 44, 45, 46, 47 are arranged spaced apart from one another in the longitudinal direction 48 of the patient receiving area 15. In an exemplary embodiment, two neighboring camera units 44, 45, 46, 47 are arranged at essentially the same distance n from one another in the longitudinal direction 48 of the patient receiving area 25. In an exemplary embodiment, the second camera unit 45 may be arranged at a distance n from the first camera unit 44. The third camera unit 46 may be arranged at a distance of n from the second camera unit 45 and at a distance 2n from the first camera unit 44. The fourth camera unit 47 may be arranged at a distance n from the third camera unit 46, at a distance 2n from the second camera unit 45 and at a distance 3n from the first camera unit 44 (FIG. 2).

The four camera units 44, 45, 46, 47 are essentially embodied as the same design. The four camera units 44, 45, 46, 47 each have at least two 2D cameras 49, 50, 51, in an exemplary embodiment they each have three 2D cameras 49, 50, 51 (FIGS. 2 and 3). As an alternative to this, each of the four camera units 44, 45, 46, 47 can each comprise one 3D camera unit.

Each of the four camera units 44, 45, 46, 47 has a defined position in the longitudinal direction 48 of the patient receiving area 25. Therefore, all 2D cameras 49, 50, 51 of one camera unit 44, 45, 46, 47 each have the defined position of the respective camera unit 44, 45, 46, 47 in the longitudinal direction 48 of the patient receiving area 15 (FIG. 2). The three 2D cameras 49, 50, 51 of one camera unit 44, 45, 46, 47 in each case are moreover arranged distributed in the circumferential direction 52 around the housing 17 surrounding the patient receiving area 15 (FIG. 3). In FIG. 3 the arrangement of the individual 2D cameras 49, 50, 51 in the circumferential direction is shown by way of example with the aid of the first camera unit 44. The arrangement of the individual 2D cameras 49, 50, 51 for the further camera units 45, 46, 47 corresponds to the first camera unit 44. In an exemplary embodiment the housing 17 surrounding the patient receiving area 15 has cutouts 53, in which a 2D camera 49, 50, 52 of one of the four camera units 44, 45, 46, 47 may be arranged in each case. As an alternative to this, the individual 2D cameras 49, 50, 51 can also be arranged on an inner wall of the housing 17 surrounding the patient receiving area 15.

An arrangement of the individual 2D cameras 49, 50, 51 in relation to one another may be the same for each of the four camera units 44, 45, 46, 47. A first 2D camera 49 of the individual camera units 44, 45, 46. 47 may be arranged in the middle at the top on the housing 17 surrounding the patient receiving area 15. A second 2D camera 50 and a third 2D camera 51 of the individual camera units 44, 45, 46, 47 are each arranged in a side area 54, 55 the housing 17 surrounding the patient receiving area 15. In an exemplary embodiment, the second 2D camera 50 may be arranged on a first side area 54 and the third 2D camera 51 on a second side area 55 lying opposite the first side area 54 and the housing 17 surrounding the patient receiving area 15. The second 2D camera 50 and the third 2D camera 51 are moreover arranged on the side areas 54, 55 at a same height 56, especially a same height in relation to a support surface of the patient table 20. Moreover, the two 2D cameras 50, 51, especially the second 2D camera 50 and the third 2D camera 51, are arranged at an essentially maximum distance from one another on the housing 17 surrounding the patient receiving area 15. Here the two 2D cameras 50, 51, especially the second 2D camera 50 and the third 2D camera 51, are arranged at approximately half the height 56 of the patient receiving area on the housing 17 surrounding the patient receiving area 15 (FIG. 3).

The individual cameras, especially the individual 2D cameras 49, 50, 51 of the four camera units 44, 45, 46, 47, as well as a fixed position, moreover, have a permanent and/or fixed field of view 57. In an exemplary embodiment, for all 2D cameras 49, 50, 51 of the four camera units 44, 45, 46, 47 have the same size of field of view 57, wherein the fields of view directly overlap (FIGS. 2 and 3).

The magnetic resonance apparatus 10 moreover has a processing unit (processor) 58, which establishes a 3D image from individual images, especially from the two-dimensional individual images, of the individual 2D cameras 49, 50, 51 of a respective camera unit 44, 45, 46, 47 (FIG. 1). Moreover, the processor 58 may be configured to create from the individual images, especially the individual 3D images, of the individual camera units 44, 45, 46, 47 and/or from the 2D images of the individual 2D cameras 49, 50, 51, an overall image of the region of the patient 16 located within the patient receiving area 15. Moreover, the processor 58 configured, with the aid of the information captured by means of the individual 2D cameras 49, 50, 51, to create a patient model. For this, the processor 58 may have the required image reconstruction software (e.g., stored therein and/or the processor 58 may be configured to access an external memory to access the software). In an exemplary embodiment, the processor 58 has an algorithm for establishing three-dimensional images, which for example may comprise a RANSAC algorithm and/or an ICP algorithm. The processor 58 may be arranged outside the scanner, especially the magnet unit 11. In an exemplary embodiment, the processor 58 can be arranged within the control room 29.

The 3D images and/or patient models established by the processor 58 are forwarded to the monitoring user interface 30 and displayed there to the medical operating personnel on the output unit 33 of the monitoring user interface 30. This enables live tracking of the patient 16 to be made available to the medical operating personnel during the magnetic resonance examination. In particular here impermissible positions of the patient 16, such as a folding of the arms, during the magnetic resonance examination, can be detected by the medical operating personnel, and where required, remedial measures can be initiated.

A second interface unit 40 of the patient interface 31 may comprise at least one illumination element 59, wherein the second interface unit 40, especially the at least one illumination element 59, may be connected to a second cable unit 36 of the data transmission unit 34 (FIG. 1). In an exemplary embodiment the second patient interface 40 has two illumination elements 59. In an alternate embodiment the second interface unit 40 can also only comprise a single illumination element 59 or also more than two illumination elements 59.

The two illumination elements 59 may each be configured as passive illumination elements 59, which are only embodied for emitting light. The passive illumination elements 59 in an exemplary embodiment are arranged on the housing 17 surrounding the patient receiving area 15. Here the passive illumination elements 59 are arranged on an inner wall of the housing 17 surrounding the patient receiving area 15. In an exemplary embodiment the passive illumination elements 59 are arranged in an area next to the patient table 20 within the patient receiving area 15 (FIGS. 1 and 3), in order to prevent the patient being blinded. In an exemplary embodiment, a passive illumination element 59 of the second interface unit 40 may be arranged on both sides of the patient table 30 in each case (FIG. 3). In an alternate embodiment the passive illumination elements 59 can also be arranged at further positions on the housing 17 surrounding the patient receiving area.

The magnetic resonance apparatus 10 moreover also has a further illumination element 61, which may comprise an active illumination element 61 (FIG. 1). The active illumination element 61 may comprise a light-generating illumination element 61, which may be arranged outside the patient receiving area 15. A transmission of light between the active, especially the light-generating illumination element 61 and the passive, especially the light-emitting illumination element 59 may be undertaken by means of the optical fibers 38 of the second cable unit 36.

The illumination elements 59, 61 may be controlled by a controller of the monitoring user interface 30 not shown in any greater detail, so that the medical operating personnel can control an illumination and/or lighting up of the patient receiving area 15 from the control room 29 by means of the monitoring user interface 30.

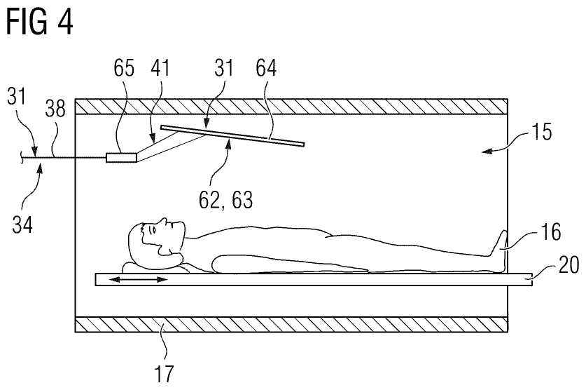

A third interface unit 41 of the patient interface 31 may comprise a visual output unit 62, wherein the third interface unit 41, especially the visual output unit 62, may be connected to a third cable unit 37 of the data transmission unit 34 (FIG. 4). The visual output unit 62 may be configured for output of information to the patient 16. For the sake of clarity, the patient receiving area 15 is only shown in FIG. 4 with the third interface unit 41 of the patient interface 31, although the patient interface 31 may comprise the three interface units 39, 40, 41. The visual output unit 62 may comprise a projection unit 63 with a projection surface 64 and an optical projection element 65. The projection surface 64 in an exemplary embodiment may comprise a mirror, which may be arranged on the housing 17 surrounding the patient receiving area 15. The projection surface 64, especially the mirror, may be arranged in the top middle on the housing 17 surrounding the patient receiving area 15, so that the projection surface 64, especially the mirror, may be arranged in a field of view of the patient 16 during the magnetic resonance examination.

The optical projection element 65 may comprise a lens and/or a lens system and/or a prism. The optical projection element 65 couples to the optical fibers 38 of the third cable unit 37. The output information may be transmitted via the optical fibers 38 of the third cable unit 37 to the patient 16 into the optical projection element 65 and from there may be projected onto the projection surface 64.

The output information for the patient 16 can be provided by the medical operating personnel, in order for example to notify or to prepare the patient 16 for a specific examination situation. Moreover, by means of the output information, the medical operating personnel can also exert a calming influence on the patient 16 during the magnetic resonance examination. The output information may comprise a remaining examination time. Moreover, the output information can also comprise instructions to the patient 16, such as breathing instructions for specific examination steps or positioning instructions, in order for example to avoid any contact between patient 16 and the housing 17 surrounding the patient receiving area 15.

The magnetic resonance apparatus 10 shown can naturally comprise further components that magnetic resonance apparatuses 10 usually have. A general way in which a magnetic resonance apparatus 10 functions is moreover known to the person skilled in the art, so that a detailed description of the further components is dispensed with here.

Although the disclosure has been illustrated and described in greater detail by the preferred exemplary embodiment, the disclosure is not restricted by the disclosed examples and other variations can be derived herefrom by the person skilled in the art without departing from the scope of protection of the disclosure.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

The various components described herein may be referred to as “modules,” “units,” or “devices.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such modules, units, or devices, as applicable and relevant, may alternatively be referred to herein as “circuitry,” “controllers,” “processors,” or “processing circuitry,” or alternatively as noted herein.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A magnetic resonance apparatus comprising:
a scanner;
a patient receiving area surrounded at least partly by the scanner;
a monitoring user interface configured to perform, by a user, monitoring and/or control of a magnetic resonance examination;
a patient interface arranged within the scanner; and
a data transceiver configured to transmit data between the patient interface and the monitoring user interface, the data transceiver including at least one cable unit, wherein, within the scanner, the at least one cable unit comprises optical fibers.

2. The magnetic resonance apparatus as claimed in claim 1, wherein the patient interface comprises at least two different interface units and the data transceiver comprises at least two cable units with optical fibers, wherein each of the at least two interface units with one of the at least two cable units is connected to the monitoring user interface and configured for data transmission.

3. The magnetic resonance apparatus as claimed in claim 1, wherein the data transmitter comprises at least one signal converter configured to convert optical signals into electronic signals and/or from electronic signals into optical signals.

4. The magnetic resonance apparatus as claimed in claim 1, wherein the data transceiver comprises a shared signal converter configured to convert optical signals into electronic signals for the at least one cable unit of the data transceiver.

5. The magnetic resonance apparatus as claimed in claim 1, wherein the patient interface comprises an interface unit including a camera system configured to monitor a patient with two or more camera units, wherein a cable unit, of the at least one cable unit, is assigned to the camera system.

6. The magnetic resonance apparatus as claimed in claim 5, wherein the two or more camera units are arranged, spaced apart from each other, in a longitudinal direction of the patient receiving area.

7. The magnetic resonance apparatus as claimed in 5, wherein the camera system comprises at least three camera units, wherein each two neighboring camera units of the at least three camera units are arranged at an equal distance in relation to each other in the longitudinal direction of the patient receiving area.

8. The magnetic resonance apparatus as claimed in claim 5, wherein each of the two or more camera units comprises a three-dimensional (3D) camera or at least two two-dimensional (2D) cameras.

9. The magnetic resonance apparatus as claimed in claim 8, wherein the at least two 2D cameras of a respective camera unit are arranged, distributed in a circumferential direction around the patient receiving area, on a housing surrounding the patient receiving area, and wherein the at least two 2D cameras of a respective camera unit each have a same position in the longitudinal direction of the patient receiving area.

10. The magnetic resonance apparatus as claimed in claim 5, wherein each of the two or more camera units comprises three two-dimensional (2D) cameras in each case, wherein the three 2D cameras of a respective camera unit are in arranged, distributed in the circumferential direction around the patient receiving area, on a housing surrounding the patient receiving area and have a same position in the longitudinal direction of the patient receiving area.

11. The magnetic resonance apparatus as claimed in claim 10, wherein a first 2D camera of the three 2D cameras is arranged in a middle at a top on the housing surrounding the patient receiving area and a second 2D camera of the three 2D cameras and a third 2D camera of the three 2D cameras are arranged, in each case, on a side area of the housing surrounding the patient receiving area, wherein the second 2D camera and the third 2D camera are arranged on opposite side areas.

12. The magnetic resonance apparatus as claimed in claim 10, wherein the second 2D camera and the third 2D camera have a position at a same height on the housing surrounding the patient receiving area and are arranged at a maximum distance in relation to each other on the housing surrounding the patient receiving area.

13. The magnetic resonance apparatus as claimed in claim 8, further comprising a processor configured to:
establish a 3D image based on two-dimensional images of the 2D cameras of a camera unit, and/or
establish a 3D overall image and/or patient model from 3D images from at least two camera units.

14. The magnetic resonance apparatus as claimed in claim 8, wherein the individual 3D cameras or the 2D cameras of the respective camera unit comprise a fixed field of view (FOV), wherein FOVs of directly neighboring camera units at least partly overlap.

15. The magnetic resonance apparatus as claimed in claim 1, wherein the patient interface comprises an interface unit with at least one illumination element, wherein a cable unit of the data transceiver is assigned to the at least one illumination element.

16. The magnetic resonance apparatus as claimed in claim 1, wherein the patient interface comprises an interface unit with a visual output unit configured to output information to the patient, wherein a cable unit of the data transceiver is assigned to the visual output unit.

17. The magnetic resonance apparatus as claimed in claim 16, wherein the visual output unit comprises a projection unit with a projection surface and an optical projection element.
